(19) United States
(12) Patent Application Publication
(10) Pub. No.: US 2005/0112089 A1
(43) Pub. Date: May 26, 2005

(54) Conjugate of biodegradable aliphatic polyester with TAT 49-57 peptide or peptide chain containing TAT 49-57 peptide and nanoparticle manufactured using the same

(76) Inventors: Ju Young Park, Yongin-si (KR); Yoon Sung Nam, Yongin-si (KR); Sang Hoon Han, Suwon-Si (KR); Ih Seop Chang, Yongin-si (KR)

Correspondence Address: HESLIN ROTHENBERG FARLEY & MESITI PC, 5 COLUMBIA CIRCLE, ALBANY, NY, 12203, US

(21) Appl. No.: 11/027,967

(22) Filed: Jan. 3, 2005

Related U.S. Application Data
(62) Division of application No. 10/185,593, filed on Jun. 28, 2002.

(30) Foreign Application Priority Data
May 17, 2002 (KR) 2002/27328

(51) Int. Cl. A61K 39/21; A61K 9/50

(52) U.S. Cl. 424/78.27; 424/188.1; 525/54.1

(57) ABSTRACT

Conjugates of a biodegradable aliphatic polyester-based polymer with Tat₄₉₋₅₇ peptide or a peptide chain containing the Tat₄₉₋₅₇ peptide, and nanoparticles manufactured using the same. Intracellular permeability of the Tat₄₉₋₅₇ peptide can be enhanced by exposing Tat peptide moieties to the surface of the nanoparticles.

### CLAIM FOR BENEFIT FOREIGN PRIORITY

This application claims priority from Korean Patent Application Number 2002/27328, filed May 17, 2002. The entire contents of the prior application is incorporated herein by reference.

### FIELD OF THE INVENTION

The present invention relates to conjugates of a biodegradable aliphatic polyester-based polymer with Tat₄₉₋₅₇ peptide or a peptide chain containing the Tat₄₉₋₅₇ peptide, and nanoparticles manufactured using the same. The term “Tat₄₉₋₅₇ peptide” as used herein refers to a peptide having the amino acid sequence of SEQ ID NO: 1. (Each of the SEQ ID NO's 8 referred to in this disclosure is detailed sheets 2-1 and 2-2 of the accompanying paper copy of the Sequence Listing.

### BACKGROUND OF THE INVENTION

The usefulness of drug delivery systems using nanoparticles has recently been studied, and special attention is paid to studies for effective manufacturing of nanostructures through various synthetic routes of polymers. Among them, studies for attaching an adhesive molecule capable of recognizing a cell to the surface of a polymer nanoparticle in order to improve the adhesion efficiency of the polymer nanoparticle to living cells have been vigorously performed. For example, these studies include solubilizing non-soluble drug to enhance its bioavailability, heightening the intracellular absorptivity of macromolecular drugs such as proteins or genes, and introducing cell recognition molecules or cell adhesive molecules into polymer nanoparticles in order to specifically deliver drugs for curing terminal diseases such as cancer, to a target cell.

Examples of the cell recognition molecule or cell adhesive molecule include sugar moiety, antibodies, peptides, etc, and as strategies for applying these molecules to drug delivery, the concept of drug-polymer prodrug conjugates are widely used. In this sense, functional polymer into which water-soluble drug and cell recognition molecule, etc., are introduced was used to deliver a drug.(H. Ringsdorf, J. Polymer Science, 51:155 (1977). Kopecek et al. demonstrates that drugs can be covalently conjugated to polymer chain by a peptide bond which is specifically cleaved by a lysosomal enzyme, after monoclonal antibodies, sugar moieties specific for liver cell, etc., are incorporated into the side chain of N-(2-hydroxypropyl)methacryl amide (HPMA) R. Duncan et al., Biochim. Biophys. Acta.,755:518 (1983); P. A. Flanagan et al., Biochim. Biophys. Acta., 39:1125 (1989); D. Putnam and J. Kopecek, J. Adv. Polymn. Sci., 122:55 (1995).

Particularly, since the 1990's, research for introducing a cell adhesive molecule into polymer nanoparticles has been earnestly active. T. Akaike et al. manufactured nanoparticles using phase separation of hydrophobic polymer and hydrophilic galactose in an aqueous solution, after synthesizing N-p-vinylbenzyl-O-beta-D-galactopyranosyl-(1,4)-D-gluconamide by incorporating galactose molecule known as having high biding force with asialoglycoprotein receptor present on the surface of hepatocytes, liver parenchymal cell, into a hydrophobic polymer chain. As a result of in vitro and in vivo experiments for these nanoparticles, it was observed that the nanoparticles are not absorbed into non-parenchymal cells such as Kupper cell in cells constituting liver tissue, but are selectively absorbed into hepatocytes (S. Tobe et al., Biochem. Biophys. Res. Commun. (1992) 184, 225; K. Kobayashi et al., Macromolecules (1997) 30, 2016). Meanwhile, M. Hashida et al. synthesized cholesten-5-yloxy-N-(4-((1-imino-2-beta-D-thiomannosylethyl)amino)butyl)formamide (Man-C4-Chol), a cholesterol derivative, by introducing a mannose receptor which belongs to C-type lectin having strong binding force to macrophages, and improved the target activity towards macrophages by incorporating it into liposomes (P. Opanasopit et al., Biochim. Biophys. Acta. (2001) 1511, 134).

In addition to the endocytosis, as it was found that macromolecules are efficiently introduced into cells through the membranes of eukaryotic cells in an energy-independent manner by a peptide responsible for import cell signaling, researches for improving the intracellular permeability of macromolecules such as proteins, liposomes, nanoparticles, etc., using a membrane-permeable protein or a peptide present on the surface of viruses have been in rapid progress (M. Lindgren et al., Trends in Pharmacological Sciences (2000) 21, 99). It is highly estimated in that these researches enhance the pharmaceutical values of macromolecules such as curative proteins or genes, which had many limitations due to low biomembrane permeability and relatively short half life in vivo. S. R. Schwarze et al. reported that a cell membrane-permeable peptide is used for delivery of a drug with high molecular weight through the blood-brain barrier which is composed of a monolayer of endothelial cells (S. R. Schwarze et al., Science (1999) 285, 1569). C. Rousselle et al. performed the study for delivering a drug through the blood-brain barrier by binding D-penetratin (all amino acids are D-isomers) having the amino acid sequence of SEQ ID NO: 2 and a membrane-permeable peptide, SynB1 having the amino acid sequence of SEQ ID NO: 3 to doxorubicin, an anticancer agent (C. Rousselle et al., Molecular Pharmacology (2000) 57, 679).

Such a cell membrane-permeable peptide is mainly derived from proteins. These peptides are largely classified into three categories:

Penetratin, a peptide derived from a homeodomain. It has the amino acid sequence of SEQ ID NO: 2. It was found in the homeodomain of Antennapedia which is a. homeoprotein of Drosophila (A. Joliot et al., Proc. Natl. Acad. Sci. U.S.A., (1991) 88, 1864). The term “homeoprotein” as used herein refers to a kind of transcription factor having about 60 amino acids which can bind to a DNA called a homeodomain.

Tat₄₉₋₅₇ peptide present between amino acids 49-57 of Tat proteins, a transcription-activating protein of human immunodeficiency virus type-1 (HIV-1) which mediates acquired immune deficiency syndrome (AIDS). It has the amino acid sequence of SEQ ID NO: 1 (P. A. Wender et al., PNAS (2000) 97, 24, 13003-13008).

Peptides based on membrane translocating sequences (hereinafter, referred to as “MTS”) or signal sequences. They are recognized by a receptor protein that helps place. proteins produced by RNA in appropriate organelle membrane in vivo. It was also found that MTS bound to nuclear localization signal (hereinafter, referred to as “NLS”) is accumulated within the cell nuclei after translocating across the cell membrane of several cells. The above mention was confirmed for MTS derived from the hydrophobic region of the signal sequence in, for example a Nuclear Transcription Factor kappa B (NF-κB), Simian virus 40 (SV40) T-antigen or K-FGF bound to NLS peptide derived from Kaposi sarcoma fibroblast growth factor 1 (hereinafter referred to as “FGF”), human beta3 integrin, HIV-1 gp41, etc. (Y. Lin et al., J. Biol. Chem. (1996) 271, 5305; X. Lin et al., Proc. Natl. Acad. Sci. U.S.A. (1996) 93, 11819; M. C. Morris et al., Nucleic Acids Res. (1997) 25, 2730; L. Zhang et al., Proc. Natl. Acad. Sci. U.S.A. (1998) 95, 9184; Chaloin et al., Biochem. Biophys. Res. Commun. (1998) 243, 601; Y. Lin et al., J. Biol. Chem. (1995) 270, 14255).

When these peptides approach a cell after binding to cargo molecules, they act as an import signal and derive intracellular translocation of the cargo molecules. M. Rojas et al. conducted a study for glutathione-S-transferase-Grb2SH2 fusion protein (41 kDa) attached by the signal chain peptide, a transport peptide, having the amino acid sequence of SEQ ID NO: 4 to examine the intracellular effect on the EGF-stimulated signaling pathway of a fusion protein comprising Grb2SH2 domains (M. Rojas et al., Nature Biotech (1998) 16, 370). S. Fawell et al. attached the amino acids 32-72 of Tat protein to RNase A, in order to detect cellular cytotoxicity through the study about inhibition of protein synthesis by regulating the efficiency of internalization (S. Fawell et al., I(1994) 91, 664). M. Rojas et al. attached the signal chain peptide having the amino acid sequence of SEQ ID NO: 4 to SHC Tyr 317 region (12 residues) in order to examine the effect on phosphorylation of Grb2 protein by the intracellular delivery of Grb2SH2 attached to peptides into SAA cells (M. Rojas et al., I (1997) 234, 675). J. Oehlke et al. attached the peptide, being an amphiphilic model peptide, having amino acid sequence of SEQ ID NO: 5 to SV40 large T antigen, in order to test the mobility of amphiphilic model peptide toward cells (J. Oehlke et al., Biochim. Biophys. Acta (1998) 1414, 127). L. Theodore et al. attached penetratin to PKC pseudo-substrate (14 residues), in order to inhibit the PKC activity of living neurocyte (T. Theodore et al., J. Neurosci. (1995) 15, 7158). S. Calvet et al. attached penetratin to FGF receptor phosphopeptide (9 residues), in order to inhibit the receptor signal system of fibroblast growth factor (hereinafter, referred to as “FGF”) in living neurocyte (S. Calvet et al., J. Neurosci. (1998) 18, 9751). M. C. Morris et al. attached MPG, a signal chain, to HIV natural primer binding site (36-mer), in order to detect intracellular delivery by a vector peptide (M. C. Morris et al., Nucleic Acid Res. (1997) 25, 2730).

B. Allinquant et al. attached penetratin to APP antisense (25-mer), in order to control the decrease of amyloidal precursor protein for the study of the effect on growth of neural spine (B. Allinquant et al., J. Cell Biol. (1995) 128, 919). S. Dokka et al. attached a signal chain peptide having the amino acid sequence of SEQ ID NO: 4 to 10 oligo nucleic acid salts, in order to study the delivery of the oligo nucleic acid salts by combining them with the synthesized import signal (S. Dokka et al., Pharm. Res. (1997) 14, 1759). M. Pooga et al. attached penetratin and transportan to galanin receptor antisense (21-mer), in order to regulate galanin receptor levels and modify pain transmission in vivo (M. Pooga et al., Nature Biotech. (1998) 16, 857).

The term “Tat peptide” as used herein refers to a part of the Tat protein chain involved in the transcription of HIV, which mediates AIDS. The Tat protein is a transcriptional activation factor which is composed of 86 to 102 amino acids depending on virus strains. The Tat protein consists of three different functional domains: an acidic amino terminal region playing an important role in transactivation, a region corresponding to amino acids 22 to 37, in which zink-finger motif is contained and to which cysteine-rich nucleic acid can be attached, and a basic region corresponding to amino acids 49 to 58 responsible for nucleus permeability. Among them, the basic region is involved in the cell adhesion of protein independently of calcium ion (S. Ruben et al., J. Virol., 63:1 (1989); B. E. Vogel et al., J.Cell Biol.,121:461 (1993).

The Tat protein is secreted by living cells and intracellularly reinternalized, like the specific homeoprotein and herpes simplex virus type I protein VP22 (HSV-1 protein VP22), etc (B. Ensoli et al., J. Virol. (1993) 67, 277). The intracellular translocation is dependent on time and concentration, and is partly inhibited in case of low temperature. Further, because chloroquine or lysosomotropic agent prevents Tat protein from decomposing and stimulates its internalization in some cells, it is proposed that Tat protein can be internalized by endocytosis (A. D. Frankel and C. O. Pabo, Cell (1988) 55, 1189). However, from the fact that cell internalization of Tat protein has low dependency on temperature (D. A. Mann and A. D. Frankel, EMBO J. (1991) 10, 1733), an alternative mechanism, especially, competitive translocation mechanism is expected to exist. For example, when a cationic polymer such as heparin or dextran sulfate is added, the intracellular translocation of Tat protein is known to decrease. Such an effect seems to be caused by competition among charged molecules on the cell membrane.

Meanwhile, it is also reported that the intracellular translocation of Tat protein is stimulated by the addition of a basic peptide such as protamine or a partial peptide of Tat protein, (amino acids 38-58). After intracellular internalization, the whole protein, or amino acids 1-86 or amino acids 37-72 of Tat protein is located in the cell nucleus. Particularly, amino acid sequence present on 48-60 is known as most effective region. Because this region contains a basic region of protein and NLS, the translocation of Tat protein into the cell or nucleus can be accomplished.

A. D. Frankel and C. O. Pabo from Johns Hopkins University Medical Center first noted the intracellular translocation of Tat protein. They found that “Tat protein” produced by HIV virus had properties of NLS localizing to the nucleus as well as translocating into the cell membrane, and that these phenomena were promoted by a low concentration of 1 nmol chloroquine (A. D. Frankel, C. O. Pabo, Cell (1988) 55, 1189). Thereafter, upon searching for the peptide region in Tat peptide responsible for membrane permeability, it was found that a site consisting of six arginines, two lysines, and one glutamine plays an important role in cell permeability, and it has the amino acid sequence of SEQ ID NO: 1.

Recently, research results reporting translocation of polymers or proteins in vivo or in vitro using Tat₄₉₋₅₇ peptide or a peptide chain containing Tat₄₉₋₅₇ peptide have been reported. To date, it has been found that at least 10 peptides derived from Tat protein are translocated into different cells. It is also known that the intracellular translocation or internalization requires only a few minutes and is not highly sensitive to temperature. It is now known that the amino acid sequence of Tat protein effective for the intracellular delivery is a amino acids 49-57.

A study for intracellular delivery by binding various cargo molecules to the above amino acid sequence has been performed. Examples of the molecules delivered by the cargo molecules include inhibitor of human papillomavirus type 16 (HPV-16), Cdk inhibitor p27^{Kip1}, p16^{INK4a}, capase-3 protein, ovalbumin to MHC class I pathway, beta-galactoxidase, etc. In addition, many studies for delivering molecules into cells have been performed using the arginine-rich amino acid sequence 48-60 of Tat protein. These molecules include DNA, macromolecules, proteins, drugs, drug delivery carriers, antigens, antibodies, hydrophilic polymers, inorganic nanoparticles, etc.

M. Lewin et al. at Massachusetts General Hospital, developed a superparamagnetic nanoparticle attached with a Tat peptide containing a short amino acid sequence 49-57 of Tat protein, which functions as a diagnostic substance to image the differentiation or distribution of precursor cells or stem cells in vivo with a high degree resolution. In this nanoparticle, 4 mer of amino acids -Gly-Tyr-Lys-Cys is attached to the carboxyl-terminal region for binding moiety of amino acids 49-57 of Tat protein, and a complex is manufactured by binding FITC, a fluorescent substance, and a nanoparticle having a diameter of 45 nm to the free —SH group of the cysteine (M. Lewin et al., Nature Biotech (2000) 18, 410).

In the results, the effective internalization of Tat peptide-modified nanoparticles into haematopoietic cells and nerve cell precursors was confirmed from in vitro experiments using CD34⁺ cells. Further, when Tat peptide-modified nanoparticles were injected intravenously into an immune deficient mouse, CD34⁺ cells originated from bone marrow were confirmed by magnetic resonance imaging.

These results indicate that some amino acid sequences, and more specifically amino acids 49-57 of Tat protein, having cell internalization function, can be attached to a synthetic polymer capable of delivering drugs. To determine whether a large water-soluble polymer such as hydrogel can be intracellularly delivered using Tat peptide, J. Kopecek et al. from Utah University bound fluorescently labeled Tat peptide to N-(2-hydroxypropyl)methacryl amide (HPMA) copolymer and intracellular delivery experiments were performed using A2780 human ovarian carcinoma cells.

As a result, it was confirmed that Tat peptide-attached HPMA is accumulated within the cells, particularly within nuclei, by a time-dependent non-endocytic pathway (A. Nori et al., 28th Proceed. of International Symposium on Controlled Release Bioactive Materials, 2001, San Diego). These results show that even in the case of a copolymer having relatively high molecular weight, intracellular delivery can be performed through binding to Tat peptide. However, because the polymer used is a hydrogel soluble in an aqueous solution, it is required to conjugate a drug to be delivered with a polymer chain. Particularly, in the case of an insoluble drug, it is difficult to attain.

### SUMMARY OF THE INVENTION

Therefore, the present invention has been made in view of the above problems, and it is an object of the present invention to provide conjugates obtainable by binding a membrane-permeable peptide chain to a polymer.

It is another object of the present invention to provide nanoparticles, whose intracellular permeability is enhanced by the use of the conjugates.

### BRIEF DESCRIPTION OF THE DRAWINGS

The above and other objects, features and other advantages of the present invention will be more clearly understood from the following detailed description taken in conjunction with the accompanying drawings, in which:

FIG. 1 shows typical Fourier transform IR spectra of the conjugate (a) produced in Example 1 and pure poly(D,L-lactic acid-co-glycolic acid) (b);

FIG. 2 shows the size distributions of nanoparticles (c) manufactured in Example 4 and the nanoparticles (d) produced in Comparative Example 1;

FIG. 3 is a transmission electron microscopic image of the nanoparticles manufactured in Example 4;

FIG. 4 shows the results of MTT cytotoxicity assay for the nanoparticles manufactured in Example 4 and the nanoparticles manufactured in Comparative Example 1;

FIG. 5a is a confocal laser scanning microscopic image showing the degree of intracellular translocation of nanoparticles manufactured in Comparative Example 1 at 37; and

FIG. 5b is a confocal laser scanning microscopic image showing the degree of intracellular translocation of nanoparticles manufactured in Example 4 at 37.

### DETAILED DESCRIPTION OF THE INVENTION

The present invention provides conjugates of a biodegradable aliphatic polyester-based polymer with Tat₄₉₋₅₇ peptide or a peptide chain containing the Tat₄₉₋₅₇ peptide; and nanoparticles manufactured using the same. That is, the present invention provides conjugates of a biodegradable aliphatic polyester-based polymer with Tat₄₉₋₅₇ peptide, or conjugates of a biodegradable aliphatic polyester-based polymer with a peptide chain containing the Tat₄₉₋₅₇ peptide; and nanoparticles manufactured using the same.

The Tat₄₉₋₅₇ peptide consists of amino acids 49-57 of Tat protein, a transcription-activating protein of human immunodeficiency virus type-1 (HIV-1) which mediates acquired immune deficiency syndrome (AIDS), and has the amino acid sequence of SEQ ID NO: 1. At least one of the Tat₄₉₋₅₇ peptides or a peptide chains containing Tat₄₉₋₅₇ peptide can be incorporated in the conjugate.

Tat₄₉₋₅₇ peptide or a peptide chain containing Tat₄₉₋₅₇ peptide is synthesized by solid phase peptide synthesis (SPPS) using amide 4-methylbenzhydrylamine hydrochloride (MBHA) resin with an ABI 433 synthesizer according to Fmoc(N-(9-fluorenyl)methoxy carbonyl)/tert-butyl method, but is not particularly limited thereto (M. Bodansky, A. Bodansky, The Practice of Peptide Synthesis; Springer: Berlin, Heidelberg, 1984, J. M. Stewart, J. D. Young, Solid Phase Peptide Synthesis, 2nd ed; Pierce Chemical Co: Rockford. Ill., 1984).

The biodegradable aliphatic polyester-based polymer is a biocompatible polymer, and is required to decompose without induction of inflammation or immune reaction, and its decomposition product is required not to harm to the human body. The most common polymer to meet the requirements is a biodegradable aliphatic polyester-based polymer having lactic acid and glycolic acid as basic units, which is approved by the U.S. FDA. Representative examples of the biodegradable aliphatic polyester-based polymer include poly(D,L-lactic acid), poly(L-lactic acid) and poly(D-lactic acid) of Formula 1, below, and poly(D,L-lactic acid-co-glycolic acid) of Formula 2, below.

The biodegradable aliphatic polyester-based polymer is at least one polymer selected from the group consisting of poly(D-lactic acid), poly(L-lactic acid), poly(D,L-lactic acid), poly(P-lactic acid-co-glycolic acid), poly(L-lactic acid-co-glycolic acid), poly(D,L-lactic acid-co-glycolic acid), poly(caprolactone), poly(valerolactone), poly(hydroxy butyrate), poly(hydroxy valerate), poly(1,4-dioxane-2-one), poly(ortho ester) and copolymers produced from the monomers corresponding to the above polymers. 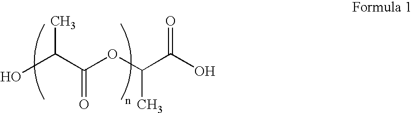
wherein
n is an integer of at least 2. 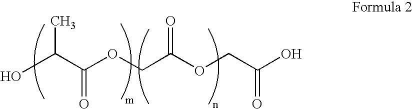
   wherein
m and n are each, independently, integers of at least 2.

In the case of poly(D,L-lactic acid-co-glycolic acid) of Formula 2, biodegradable polymer having various decomposition lifetimes can be obtained by controlling the ratio of monomers of lactic acid and glycolic acid, or changing the synthesizing pathway of polymer. Such a biodegradable aliphatic polyester-based polymer has been used as a carrier for drug delivery or a suture for operation for a long time, and already demonstrated its biocompatibility. Meanwhile, the weight average molecular weight of the biodegradable aliphatic polyester-based polymer is in the range of 500 to 100,000, preferably 5,000 to 50,000 in order to achieve good effect on the production of nanoparticles, but is not particularly limited to these ranges.

Tat₄₉₋₅₇ peptide or a peptide chain (A) containing Tat₄₉₋₅₇ peptide, and the biodegradable aliphatic polyester-based polymer (B) may be constituted as A-B type or A-B-A type, but are not particularly limited thereto. First, carboxylic groups and hydroxyl groups present on both termini of the biodegradable aliphatic polyester-based polymer may be substituted with different functional groups so as to promote covalent bonding. Subsequently, the substituted termini of the polymer are reacted with termini of the Tat₄₉₋₅₇ peptide or the termini of peptide chain containing Tat₄₉₋₅₇ peptide to obtain the above constitution. For example, a conjugate of poly(D,L-lactic acid-co-glycolic acid) with the Tat₄₉₋₅₇ peptide or peptide chain containing Tat₄₉₋₅₇ peptide can be synthesized through the covalent bonding of poly(D,L-lactic acid-co-glycolic acid) substituted with maleimide and Tat peptide having thiol-substituted termini.

In the present invention, the covalent bonding can be formed by the addition of a base, a linker or a multiligand compound between the biodegradable aliphatic polyester-based polymer and the Tat₄₉₋₅₇ peptide or peptide chain containing Tat₄₉₋₅₇ peptide, but is not particularly limited thereto.

The present invention also relates to nanoparticles manufactured using the conjugate. At this time, the smaller the average size of the nanoparticles, the more preferable it is in view of the stability of colloid. For example, the average diameter of the nanoparticle is not more than 1,000 nm, preferably not more than 300 nm. Membrane-permeability of Tat peptide can be taken effectively by exposing Tat peptide moieties on the surface of the nanoparticles according to the present invention, which results in enhanced intracellular permeability.

Methods for manufacturing the nanoparticles according to the present invention include the followings, but are not particularly limited thereto: sonicating after directly dispersing the polymer in an aqueous solution, extracting organic solvent with an excess of water or evaporating organic solvent after dispersing or dissolving the polymer in organic solvent, evaporating solvent under vigorous stirring condition by the use of a homogenizer or a high pressure emulsifier after dispersing or dissolving the polymer in organic solvent, dialyzing with an excess of water after dispersing or dissolving the polymer in organic solvent, adding water slowly after dispersing or dissolving the polymer in organic solvent, manufacturing using supercritical fluid, etc. (T. Niwa et al., J. Pharm. Sci. (1994) 83, 5, 727-732; C. S. Cho et al., Biomaterials (1997) 18, 323-326; T. Govender et al., J. Control. Rel. (1999) 57, 171-185; M. F. Zambaux et al., J. Control. Rel. (1998) 50, 31-40).

Examples of the organic solvents which can be used in the manufacture of the nanoparticles according to the present invention include acetone, dimethylsulfoxide, dimethylformamide, N-methylpyrrolidone, dioxane, tetrahydrofuran, ethyl acetate, acetonitrile, methyl ethyl ketone, methylene chloride, chloroform, methanol, ethanol, ethyl ether, diethyl ether, hexane or petroleum ether. At this time, the solvents can be used alone or in combination.

Further, the nanoparticles according to the present invention can be used as a drug delivery system with an improved bioavailability in vivo by introducing a specific drug therein.

### EXAMPLES OF THE PRINCIPLES OF THE INVENTION

The present invention will now be described in more detail with reference to the following Examples, Comparative Examples and Experimental Examples. However, materials, agents, costs, operations, etc., used may be changed by those skilled in the art without departing from the true spirit and scope of the invention. Accordingly, these examples are given by way of illustration and not of limitation.

### EXAMPLE 1

### Conjugate of poly(D,L-lactic acid-co-glycolic acid) and a Peptide Chain Containing Tat₄₉₋₅₇ Peptide

A peptide chain containing Tat₄₉₋₅₇ peptide was synthesized by solid phase peptide synthesis (SPPS) using amide 4-methylbenzhydrylamine hydrochloride (MBHA) resin with an ABI 433 synthesizer according to Fmoc(N-(9-fluorenyl)methoxycarbonyl)/t-butyl method, and then purified by reverse high performance liquid chromatography (purity: greater than 90%). The molecular weight was determined to be 1846 by mass spectroscopy (Agilent 1100 series). These results confirmed that the peptide having the amino acid sequence of SEQ ID NO: 6, which contained chain contains Tat₄₉₋₅₇ peptide and was added with a Gly-Tyr-Lys-Cys peptide consisting of 4 amino acids as a linker, was synthesized.

The conjugate of poly(D,L-lactic acid-co-glycolic acid) and the peptide chain containing Tat₄₉₋₅₇ peptide was synthesized through the covalent bonding of poly(D,L-lactic acid-co-glycolic acid) substituted with maleimide and the Tat peptide having thiol-substituted termini. The procedure is as follows: 80 ml of anhydrous 1,4-dioxane, 10 g of poly(D,L-lactic acid-co-glycolic acid) and 0.2 ml of triethylamine (TEA) were added to a reaction vessel, and the mixture was stirred to completely dissolve. To the reaction vessel a mixture of 1,3-dicyclohexyl carbodiimide (DCC) and N-hydroxysuccinimide (NHS) was added to activate the carboxylic groups in the main chain of the polymer.

At this time, the molar ratio of carboxylic groups, dicyclohexylcarbodiimide and N-hydroxysuccinimide was 1:2:2. The mixture was stirred at room temperature, 1 atm under nitrogen atmosphere for 4 hours. 200 mg of hexamethylene diamine dissolved in 10 ml of anhydrous 1,4-dioxane was added to the reaction vessel and then stirred for 2 hours. The solution, which is obtained by filtration through a nylon filter with a pore size of 0.45 lm, the reaction mixture was subjected to precipitation with anhydrous diethyl ether, and the ether was removed to obtain a white solid. The solid reactant was again added to methylene chloride to dissolve the remaining reactant, reaction agents and byproducts. From this mixture, only the synthesized polymer was precipitated and collected. The above procedure was repeated three times to further purify the polymer. The purified polymer was dried under vacuum.

The polymer, poly(D,L-lactic acid-co-glycolic acid) having amine groups at the termini, thus obtained was dissolved in methylene chloride, and 1.5 times excess moles of N-succinimidyl 4-(4-maleimidophenyl)-butyrate was added thereto to derive maleimide to the termini of poly(D,L-lactic acid-co-glycolic acid). The synthesized polymer was precipitated with anhydrous diethyl ether, purified in accordance with the above precipitation method, and dried under vacuum. 3 ml of dimethylsulfoxide (DMSO) and 100 mg of polymer thus synthesized were added to a reaction vessel, and stirred to completely dissolve. 5 mg of peptide chain having the amino acid sequence-of SEQ ID NO: 6 dissolved in 400 μl of reaction buffer (83 mM sodium phosphate buffer, 0.1 M EDTA, 0.9 M sodium chloride, 0.02% sodium azide, pH 7.2, with stabilizer) was added to the mixture, and then reacted at room temperature for at least 6 hours. After the reaction, the title compound obtained was purified by dialysis using cellulose membrane against distilled water, and lyophilized.

### EXAMPLE 2

### Conjugate of poly(D,L-lactic acid) and a Peptide Chain Containing Tat₄₉-57 Peptide

The title compound was produced in the same manner as in Example 1, except that poly(D,L-lactic acid) was used instead of poly(D,L-lactic acid-co-glycolic acid).

### EXAMPLE 3

### Conjugate of poly(L-lactic Acid) and a Peptide Chain Containing Tat₄₉₋₅₇ Peptide

The title compound was produced in the same manner as in Example 1, except that poly(L-lactic acid) was used instead of poly(D,L-lactic acid-co-glycolic acid).

### EXAMPLE 4

### Nanoparticles Using Conjugate of poly(D,L-lactic acid-co-glycolic acid) and a Peptide Chain Containing Tat₄₉₋₅₇ Peptide

Nanoparticles according to the present invention were manufactured in accordance with phase inversion method. 100 mg of conjugate produced in Example 1 was dissolved in 10 ml of acetone, and then slowly added to 100 ml of phosphate buffer solution containing 0.5% w/v polyvinyl alcohol (PVA, 88% hydrolyzed, Mw of 25,000) with rapid stirring. Conjugate of poly(D,L-lactic acid-co-glycolic acid) and peptide chain containing fluorescently labeled Tat₄₉₋₅₇ peptide (5% by weight) in acetone was used to produce a fluorescently labeled polymer nanoparticle.

Meanwhile, the fluorescently labeled conjugate was produced in accordance with the following procedure. First, 10 g of the conjugate produced in Example 1 was subjected to esterification by 500 mg of dicyclohexylcarbodiimide and 300 mg of N-hydroxysuccinimide to activate carboxyl groups of the conjugate and then covalently bound to primary amine groups of fluorescein amine. The coupling reaction between the activated conjugate and the fluorescein amine was performed at room temperature under nitrogen atmosphere for 10 hours after adding 0.5 mg of triethylamine thereto. The dicyclourea precipitated as a byproduct was removed by filtration. The fluorescently labeled conjugate was precipitated with anhydrous diethyl ether, and purified in accordance with the above precipitation method.

### EXAMPLE 5

### Nanoparticles Using Conjugate of poly(D,L-lactic acid) and a Peptide Chain Containing Tat₄₉₋₅₇ Peptide

The title compound was manufactured in the same manner as in Example 4, except that the conjugate produced in Example 2 was used instead of the conjugate produced in Example 1.

### EXAMPLE 6

### Nanoparticles Using Conjugate of poly(L-lactic acid) and a Peptide Chain Containing Tat₄₉₋₅₇ Peptide

The title compound was manufactured in the same manner as in Example 4, except that the conjugate produced in Example 3 was used instead of the conjugate produced in Example 1.

### COMPARATIVE EXAMPLE 1

### Nanoparticles of poly(D,L-lactic acid-co-glycolic acid)

The title compound was manufactured in the same manner as in Example 4, except that pure poly(D,L-lactic acid-co-glycolic acid) was used instead of the conjugate produced in Example 1.

### EXPERIMENTAL EXAMPLE 1

### Confirmation of the Conjugation of a Biodegradable Aliphatic Polyester-Based Polymer and a Peptide Chain Containing Tat₄₉₋₅₇ Peptide

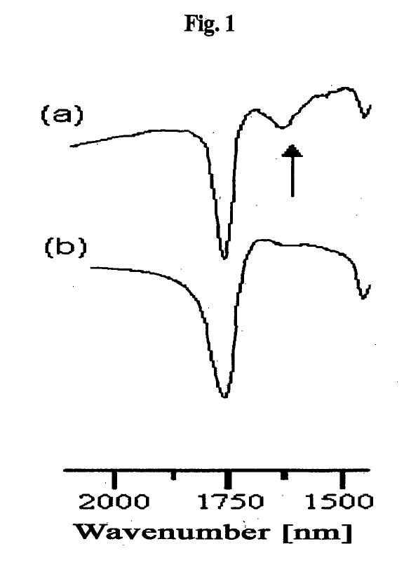

Confirmation of the conjugates according to the present invention was performed using an infrared spectrometer. FIG. 1 shows typical Fourier transform IR spectra of the conjugate (a) produced in Example 1 and pure poly(D,L-lactic acid-co-glycolic acid) (b). In the case of the conjugate (a) produced in Example 1, an amine-specific peak in the vicinity of 1656 cm-i was observed, in addition to an ester-specific peak in the vicinity of 1750 cm⁻¹. The presence of these peaks indicates that the peptide chain containing Tat₄₉₋₅₇ peptide was covalently conjugated to poly(D,L-lactic acid-co-glycolic acid).

### EXPERIMENTAL EXAMPLE 2

### Analysis of Nanoparticles

Surface potential of the nanoparticles manufactured in Example 4 and Comparative Example 1 was measured using Zetasizer 3000HS (Malvern, UK). Surface potential was −7.8 mV for the nanoparticles manufactured in Comparative Example 1, while −0.9 mV for the nanoparticles manufactured in Example 4. This suggests that the peptide chain containing cationic lysine- and arginine-rich Tat₄₉₋₅₇ peptide orients toward the surface of nanoparticles, thereby increasing the surface potential.

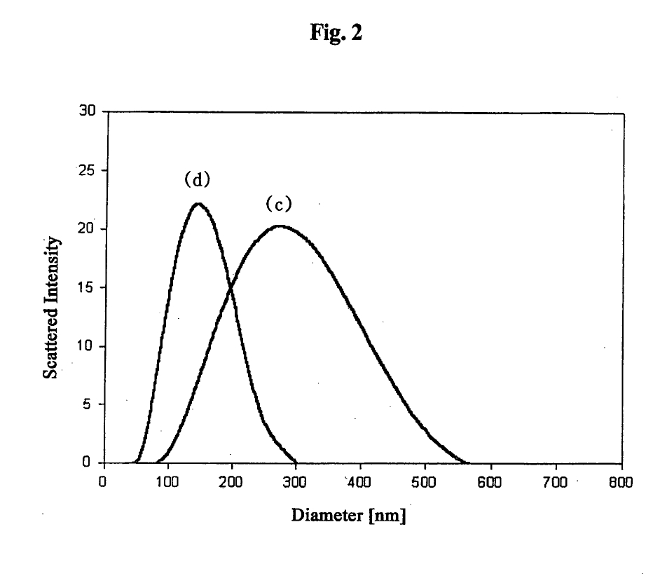

The average particle sizes of the nanoparticles manufactured in Examples 4 to 6, and Comparative Example 1 were determined in accordance with a dynamic light scattering method (Zetasizer 3000HS, Malvern, UK). The scattering angle was fixed to an angle of 90°, and the experiment was carried out at 25. The hydrodynamic particle diameter was calculated by the Contin method. The results are shown in Table 1 and illustrated graphically in FIG. 2.

As can be seen from the results, the average particle size of the nanoparticles (c) manufactured in Examples 4 to 6 is larger than that of the nanoparticle (d) manufactured in Comparative Example 1. This is thought to be resulting from the fact that the nanoparticles of pure poly(D,L-lactic acid-co-glycolic acid) have stronger hydrophobicity than the nanoparticles introduced by the peptide chain containing Tat₄₉₋₅₇ peptide, whereby forming a more compact nanostructure by their hydrophobic interaction.

TABLE 1

| | Conjugate used to | Average diameter |
|---|---|---|
| | manufacture nanoparticle | of particle |
| Example 4 | Conjugate manufactured in Example 1 | 238 nm |
| Example 5 | Conjugate manufactured in Example 2 | 220 nm |
| Example 6 | Conjugate manufactured in Example 3 | 250 nm |
| Comparative | Poly(D,L-lactic acid-co-glycolic acid) | 128 nm |
| Example 1 | | |

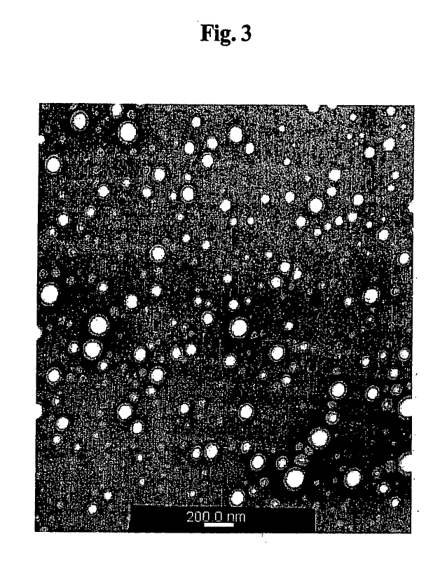

In addition, the shapes and the size distribution of the nanoparticles were observed using transmission electron microscopy (TEM, JEOL 2010). The test pieces were prepared by depositing one drop of 1 g/L nanoparticles dispersed in PBS onto a 100 mesh copper grid coated with carbon, and 1 minute after deposition, staining with 2% uranyl acetate solution. FIG. 3 is a transmission electron microscopic image of nanoparticles manufactured in Example 4. This shows that the nanoparticles have a discrete spherical morphology.

### EXPERIMENTAL EXAMPLE 3

### Cytotoxicity Assay Through Cell Culture

The cytotoxicity of the nanoparticles manufactured in Example 4 and Comparative Example 1 was evaluated using HaCaT (human corneous cell line) and HS-68 (human fibroblast cell line). The two cells were added to a cell culture medium (Dulbecco's modified Eagle's medium; hereinafter, referred to as “DMEM”) supplemented with 1% by volume antibiotics (streptomycin, 10,000,ug /ml; penicillin, 10,000 IU/ml) and 10% by volume fetal bovine serum (hereinafter, referred to as “FBS”) and incubated in an incubator filled with humidified air containing 5% CO₂ at 37.

The two cells with 75% cell density in 96-well flat-bottomed plates were incubated with 1.5-50 μg/ml nanoparticles in 100 μl culture medium for 1 hour. Then, 10% by volume FBS was added thereto and incubated for an additional 48 hours. Thereafter, 3-(4,5-dimethylthiazol-2-yl)-2,5-diphenyl tetrazolium bromide (hereinafter, referred to as “MTT”) and lactate dehydrogenase (hereinafter, referred to as “LDH”) analyses were performed to evaluate cytotoxicity.

After MTT was added to cells incubated in 96-well plates to a final concentration of 500 ug/ml and the cells were incubated at 37 for 4 hours, 100 μl of acidic isopropanol (0.04 N HCl in isopropanol) was added to each well and then mixed to dissolve dye material converted by living cells. ELISA plate reader (ELx800, Bio-TEK Instr. Inc.) was used to determine the absorbance of the converted dye material in each cell of 96-well plates at a wavelength of 550 um. The MTT analysis standard curve was calculated by analyzing the relation of change in absorbance with respect to the number of living cells, after different numbers of cells were added to each well of 96-well plates, then incubating the cells in accordance with the above method, and followed by performing the MTT assay. The cytotoxicity assay of nanoparticles manufactured in Example 4 through the MTT assay was presented as % of living cells.

The amount of LDH eluted to the cell culture medium was measured using Cyto Tox 96 Non-Radioactive Cytotoxicity Assay kit (Promega, Madison, Wis., USA). The debris of dead cells was separated by centrifuging the culture medium at a speed of 250 g for 4 minutes. After the centrifugation, 50 μl of supernatant was added to each well of 96-well plates. Subsequently, 50 μl of substrate solution was added thereto and left at room temperature for 30 minutes. To stop the reaction of the eluted LDH and substrate solution, 50 μl of 1.0 M acetic acid was added. The absorbance at 492 nm of the samples in each well was determined using an ELISA plate reader.

The eluted LDH (%) was calculated using the following relation:
Eluted LDH (%)=(LDH eluted from the damaged cells (experimental group)/maximum LDH eluted from all cells after treating with lysis solution)×100

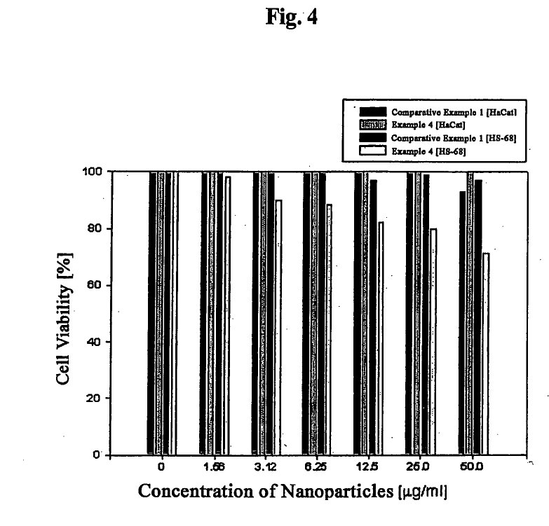

FIG. 4 shows the results of MTT cytotoxicity assay of nanoparticles manufactured in Example 4 and nanoparticles manufactured in Comparative Example 1. These results show that there is no significant difference in cytotoxicity between the nanoparticles introduced by peptide chain containing the Tat₄₉₋₅₇ peptide and the nanoparticles containing no peptide chain.

### EXPERIMENTAL EXAMPLE 4

### Intracellular Translocation of Nanoparticles of a Conjugate of poly(D,L-lactic acid-co-glycolic acid) and a Peptide Chain Containing Tat₄₉₋₅₇ Peptide

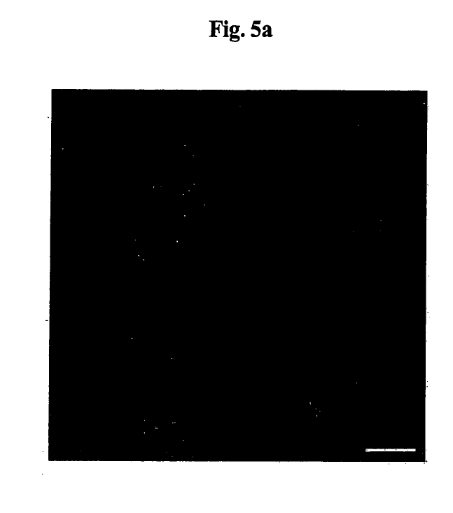

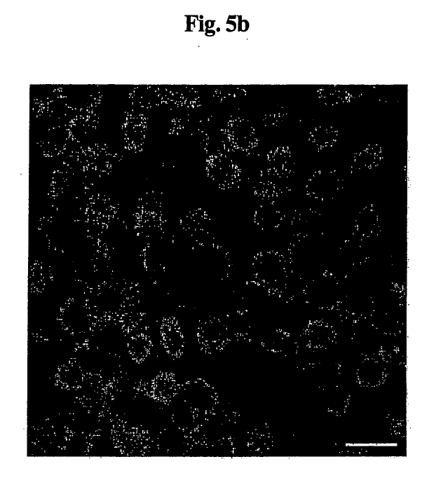

Intracellular translocation of the nanoparticies manufactured in Examples 4 and 5 was confirmed using HaCaT cells, on which the nanoparticles were confirmed to have little or no effect on cell viability by Experimental Example 3, by the use of a confocal microscope (Radiance 2000/MP, Bio-rad). HaCaT cells grown in transparent 35 mm Delta T culture dishes (0.15 mm thick) were incubated in 1 ml DMEM culture medium supplemented with 1% by volume antibiotics and fluorescently labeled polymer nanoparticles (concentration=50/ml) for 1 hour under two conditions: air containing 5% C0₂ at 37, and air at 4. After culturing in two different conditions, respectively, the cells were washed three times with 1 ml of phosphate buffer solution, 1 ml DMEM culture medium was added to the culture dishes again, and the fluorescence of dyed cells was observed. The results are shown in FIG. 5.

FIG. 5a is a confocal laser scanning microscopic image showing the degree of intracellular translocation of nanoparticles manufactured in Comparative Example 1 at 37 (each scale interval is 10), and FIG. 5b is a confocal laser scanning microscopic image showing the degree of intracellular translocation of nanoparticles manufactured in Example 4 at 37 (each scale interval is 10).

As can be seen from the figures, in the case of nanoparticles of pure poly(D,L-lactic acid-co-glycolic acid), no intracellular translocation of the nanoparticles was observed; whereas in the case of the nanoparticles manufactured in Example 4, the nanoparticles were permeated through cell membranes and translocated into cells. Therefore, intracellular translocation of nanoparticles can be enhanced by introducing peptide chain containing Tat₄₉₋₅₇ peptide to nanoparticles.

We observed that intracellular permeability of Tat₄₉₋₅₇ peptide can be enhanced by exposing Tat peptide moieties on the surface of the nanoparticles according to the present invention. Accordingly, nanoparticles according to the present invention can eliminate the disadvantages of polymer nanoparticles according to the prior art by covalently conjugating Tat₄₉₋₅₇ peptide or a peptide chain containing Tat₄₉₋₅₇ peptide, which has high biomembrane permeability, at the termini of polymer. Further, the nanoparticles according to the present invention are expected to be useful as an efficient drug delivery system with an improved bioavailability in vivo when a drug is included therein.

1-6. (canceled)

7. A nanoparticle manufactured using a conjugate comprising a biodegradable aliphatic polyester-based linear polymer covalently linked with either a Tat₄₉₋₅₇ peptide of SEQ ID NO. 1 or a peptide chain containing the Tat₄₉₋₅₇ peptide of SEQ ID No. 1, wherein the biodegradable aliphatic polyester-based polymer is at least one polymer selected from the group consisting of poly(D-lactic acid), poly(L-lactic acid, poly (D,L-lactic acid). poly(D-lactic acid-co-glycolic acid), poly(L-lactic acid-co-glycolic acid), poly(D,L-lactic acid-co-glycolic acid), poly(caprolactone), poly(valerolactone), poly(hydroxy butyrate), poly(hydroxy valerate), poly(1,4-dioxane-2-one), poly(ortho ester) and copolymers produced from the monomers corresponding to the above polymers.

8. (canceled)

9. The nanoparticle of claim 7 wherein the biodegradable aliphatic polyester-based polymer is at least one polymer selected from the group consisting of poly(D-lactic acid), poly(L-lactic acid, poly (D,L-lactic acid), poly(D-lactic acid-co-glycolic acid), poly(L-lactic acid-co-glycolic acid) and poly(D,L-lactic acid-co-glycolic acid).

10. The nanoparticle of claim 7 wherein the biodegradable aliphatic polyester-based polymer has a weight average molecular weight of from 500 to 100,000.

11. The nanoparticle of claim 7 wherein the conjugate has the structure A-B or A-B-A, wherein A is the Tat₄₉₋₅₇ peptide or a peptide chain containing the Tat₄₉₋₅₇ peptide and B is the biodegradable aliphatic polyester-based polymer.

12. The nanoparticle of claim 7 wherein a base, linker or multiligand compound is added between the biodegradable aliphatic polyester-based polymer and the Tat₄₉₋₅₇ peptide or peptide chain containing the Tat₄₉₋₅₇ peptide.

13. The nanoparticle as set forth in claim 7, which has an average size diameter not more than 1,000 nm.

14. The nanoparticle as set forth in claim 8, which has an average size diameter not more than 1,000 nm.

15. The nanoparticle as set forth in claim 9, which has an average size diameter not more than 1,000 nm.

16. The nanoparticle as set forth in claim 10, which has an average size diameter not more than 1,000 nm.

17. The nanoparticle as set forth in claim 11, which has an average size diameter not more than 1,000 nm.

18. The nanoparticle as set forth in claim 12, which has an average size diameter not more than 1,000 nm.


SEQUENCE LISTING (6 sequences)

SEQ ID NO: 1 - protein, 9 aa, Human immunodeficiency virus type 1
RKKRRQRRR

SEQ ID NO: 2 - protein, 16 aa, Drosophila Antennapedia
RQIKIWFQNRRMKWKK

SEQ ID NO: 3 - protein, 18 aa, Artificial Sequence
  peptide derived from SynB1 of protegrins
RGGRLSYSRRRFSTSTGR

SEQ ID NO: 4 - protein, 16 aa, Artificial Sequence
  peptide derived from the src homology 2 (SH2) domain of Grb2
AAVALLPAVLLALLAP

SEQ ID NO: 5 - protein, 18 aa, Artificial Sequence
  amphiphilic model peptide
KLALKLALKALKAALKLA

SEQ ID NO: 6 - protein, 14 aa, Artificial Sequence
  peptide derived from Tat49-57 peptide
GRKKRRQRRRGYKC